(19) United States
(12) Patent Application Publication
(10) Pub. No.: US 2004/0137089 A1
(43) Pub. Date: Jul. 15, 2004

(54) Skin treatment

(76) Inventor: Etsuko Dinan, Suffolk (GB)

Correspondence Address: Daniel A Monaco, Drinker Biddle & Reath, One Logan Square, 18th and Cherry Streets, Philadelphia, PA, 19103-6996, US

(21) Appl. No.: 10/474,074

(22) PCT Filed: Apr. 4, 2002
(86) PCT No.: PCT/GB02/01597

(30) Foreign Application Priority Data
Apr. 6, 2001 (GB) 0108671.9

(51) Int. Cl. A61K 35/78

(52) U.S. Cl. 424/744; 424/769

(57) ABSTRACT

This invention relates to a skin treatment. The skin treatment is particularly but not exclusively for use in medical and cosmetic treatment of the human skin. The treatment includes alcohol spirit, glycerine and extract of aloe vera or kiddachi aloe. Alternatively, the treatment includes alcohol spirit and a source of vitamin B₁₇. Preferably, the alcohol spirit is chosen from the group including barley, rice, potato spirit or sake. Preferably, it is a low class distilled spirit. Additional preferred ingredients include seed and/or leaf, extract, citrus fruit extract and tea leaf extract.

This invention relates to a skin treatment. The skin treatment is particularly but not exclusively for use in medical and cosmetic treatment of the human skin.

Human skin naturally contains moisture to give the skin elasticity and to protect it from the environment.

However, skin can become dry, flaky, irritated, inflamed and sore for a variety of reasons including, for example, exposure to extreme weather conditions, allergic reactions to substances which come into contact with the skin, rough treatment of the skin, and skin diseases or disorders such as dermatitis, eczema and psoriasis.

Skin also becomes dry naturally with age and as a result wrinkles and fine lines start to appear.

Conventionally, dry skin is treated by topical application of a moisturiser, having oil and water as the principal ingredients. Various specialised chemical treatments are available for skin diseases and disorders, such as cortisone lotions for inflammation and dermatitis

Within the cosmetic and beauty care industries there is a desire to move away from conventional methods toward more natural methods. Natural ingredients are usually less expensive and the general public would prefer to use treatments containing natural products.

Fine lines, wrinkles and other signs of ageing are generally thought of as undesirable and the public are concerned with delaying the onset of these signs for as long as possible.

Natural ingredients have been used in lotions and tonics to treat dryness of the skin. These include the use of aloe vera for treatment of sunburn and to promote wound healing. Aloe vera also has moiturising and emollient properties.

Chinese medical literature reports that other natural ingredients can be beneficial to poor skin conditions, such as ulon Tea Leaves, which are rich in vitamin C and Lemon Grass, which is a known germicide.

These known compositions often are not fully absorbed into the skin and so the benefits obtained are limited to a temporary softening of only the outermost epidermal layers.

According to the present invention there is provided a skin treatment having the components:

(a) alcohol spirit,

(b) glycerine, and

(c) an extract of aloe vera or kiddachi aloe.

According to a further aspect of the invention there is provided a skin treatment comprising alcohol spirit and a source of vitamin B₁₇.

Preferably the alcohol spirit is barley, rice or potato spirit.

In one embodiment the alcohol spirit is sake in further embodiments the alcohol spirit is shochu, which may be 35% alcohol by volume, or vodka or white whisky.

Preferably the distilled spirit is a low class distilled spirit.

Preferably the aloe vera extract is aloe vera gel.

The skin treatment may further comprise a seed and/or leaf extract.

Preferably the source of vitamin B₁₇ is a seed and/or leaf extract, most preferably from loquat leaves.

Optionally the seed and/or leaf extract comes from the seeds and/or leaves of one or more of the group consisting of prune, plum and sesame.

The skin treatment may further comprise citrus fruit extract.

Preferably the citrus fruit extract is lemon extract.

Preferably the lemon extract is lemon peel.

The skin treatment may further comprise tea leaf extract.

Preferably the tea leaf extract is obtained from ulon tea leaves.

The skin treatment may further comprise lemon grass extract.

Preferably the lemon grass extract is obtained from lemon grass sticks.

In one embodiment the components are present in the following proportions:

| | |
|---|---|
| alcohol spirit | 45%-94% |
| glycerine | 1%-40% |
| aloe vera | 3%-25% |
| lemon peel | 1%-8%. |

More preferably the skin treatment has the following composition:

| | |
|---|---|
| barley spirit | 60%-90% |
| glycerine | 2%-40% |
| aloe vera | 4%-6% |
| lemon peel | 4%-6%. |

Alternatively the lemon extract may be lemon oil. Suitably the skin treatment comprises 0.01%-2% lemon oil.

In an alternative embodiment the components are present in the following proportions:

| | |
|---|---|
| alcohol spirit | 70%-90% |
| glycerine | 1%-10% |
| aloe vera | 3%-15% |
| ulon tea leaves | 1%-5% |
| lemon grass extract | 0.01%-1%. |

More preferably the skin treatment has the following composition:

| | |
|---|---|
| alcohol spirit | 83%-88% |
| glycerine | 3%-8% |
| aloe vera | 5%-9% |
| ulon tea leaves | 2%-4% |
| lemon grass extract | 0.01%-1%. |

Any part of the aloe vera plant may be used, for example, the leaves or the stalks. Similarly parts of the lemon other than the peel may be used.

Lemon petitgrain oil may also be present in the skin tonic.

Any citrus fruit having the desired properties may be used. Mention may be made of orange or lime fruit extracts.

The skin treatment of the present invention may contain other compounds. These compounds may include perfume and/or colouring compounds.

The following non-limiting examples illustrate methods of obtaining the skin treatment of the present invention.

### EXAMPLE 1

Ingredients

1 litre of low class barley distilled spirit

500 ml of glycerine

50 g aloe vera leaves

50 g lemon peel

Method

The lemon peel is obtained by washing approximately 5-6 lemons and peeling them. Care is taken not to peel any of the pith of the lemon

The aloe vera leaves are washed then finely sliced and the aloe vera and lemon peel are placed in a suitable container.

The glycerine and the spirit are added and the container is covered.

The covered container is left in a cool dark location for approximately 2-3 months, checking occasionally.

At the end of this period, the mixture is removed from the container and sieved.

In a skin treatment made by the above method, components were found to be present in approximately the following percentages by volume by analytical and consulting chemists, Analytical Investigations:

| | Stated | Found |
|---|---|---|
| Component | Formulation | Formulation |
| Glycerine v/v % | 35.7 | 36.1 |
| Spirit v/v % | 64.3 | 65.2 |
| Aloe Vera w/v % | 5.0 | 5.2 |
| Lemon w/v % | 5.0 | 4.9 |

Instead of lemon peel, it is possible to use a small quantity of lemon oil, for example so that approximately 1% of the final composition is lemon oil.

It may be possible to substitute the lemon peel by the peel of other fruits with the desired properties.

The barley spirit can be substituted for rice or potato spirit.

The spirit can be obtained by distilling the alcohol from the Japanese liquor, sake.

The aloe Vera leaves may be substituted for 50 g of aloe vera gel, as is commonly available.

### Example 2

Ingredients

1.5 litres of sake

50 g of glycerine

150 g aloe vera leaves

50 g ulon tea leaves

1.5 lemon grass sticks

The ingredients are added together by the same method as used in Example 1, except in this Example the mixture is only left for one month in a cool dark location.

### Example 3

Ingredients

200 g loquat leaves

40 g aloe vera

shochu (35% by volume)

(preferable—40 g green tea leaves)

Whereas the above ingredients represent a specific embodiment the relative amounts are not required to be exact and the following ranges of ingredients may be used.

50-500 g loquat leaves

5-80 g aloe vera

(15-50% by volume) shochu

5-80 g green tea leaves

The amounts used may depend on the required characteristics of the resulting composition.

The loquat and aloe vera are loosely packed into a container which is then filled with the shochu spirit. The mixture is left for one to two months in a cool dark location. This process can be speeded up by heating, preferably under reflux, if necessary. Thereafter the liquid is decanted and is ready for use.

The final composition is in a stabilised state, having been stabilised by the alcohol spirit, and can be stored for future medical and cosmetic uses. It is effective in treating eczema, burns, cuts, insect bites, ulcers, thrush and other complaints.

In general, 5-80 g of aloe vera can be used effectively.

It is thought that the presence of the alcohol spirit may aid skin penetration of the aloe vera and loquat constituents.

Modifications and improvements may be made without departing from the scope of the invention.

1. A skin treatment comprising:
(a) alcohol spirit,
(b) glycerine, and
(c) an extract of aloe vera or kiddachi aloe.

2. A skin treatment comprising alcohol spirit and a source of vitamin B₁₇.

3. A treatment as claimed in claim 1 or 2 wherein the alcohol spirit is barley, rice or potato spirit, or sake or shochu, which may be 35% alcohol by volume, or vodka or white whisky.

4. A skin treatment as claimed in any of the proceeding claims wherein the distilled spirit is a low class distilled spirit.

5. A skin treatment as claimed in any of the preceding claims wherein the aloe vera extract is aloe vera gel.

6. A skin treatment as claimed in any preceding claim further comprising a seed and/or leaf extract.

7. A treatment as claimed in any preceding claim wherein the source of vitamin B₁₇ is a seed and/or leaf extract, most preferably from loquat leaves.

8. A skin treatment as claimed in any preceding claim further comprising citrus fruit extract.

9. A skin treatment as claimed in any preceding claim further comprising tea leaf extract.

10. A skin treatment as claimed in any preceding claim further comprising lemon grass extract

11. A skin treatment comprising

| | |
|---|---|
| alcohol spirit | 45%-94% |
| glycerine | 1%-40% |
| aloe vera | 3%-25% |
| lemon peel | 1%-8%. |

12. A skin treatment comprising

| | |
|---|---|
| alcohol spirit | 70%-90% |
| glycerine | 1%-10% |
| aloe vera | 3%-15% |
| ulon tea leaves | 1%-5% |
| lemon grass extract | 0.01%-1%. |
